(19) United States
(12) Patent Application Publication
(10) Pub. No.: US 2007/0048241 A1
(43) Pub. Date: Mar. 1, 2007

(54) Emulsifying system

(75) Inventors: Patrick Obukowho, Fords, NJ (US); Kevin Gallagher, Lincroft, NJ (US); Abel G. Pereira, Bridgewater, NJ (US); Robert Comber, Doylestown, PA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Croda, Inc., Edison, NJ (US)

(21) Appl. No.: 11/215,687

(22) Filed: Aug. 30, 2005

(51) Int. Cl. A61K 8/37

(52) U.S. Cl. 424/70.31

(57) ABSTRACT

The present invention is an oil-in-water emulsion having a unique emulsifier system which is useful in high oil content products and products susceptible to phase inversion.

### BACKGROUND OF THE INVENTION

The formulation of hair and skin care products is often challenging. One of those challenges is the creation of stable emulsified products. Emulsions tend to have very good properties in terms of their feel, their ability to hold disparate components and the like. However, the manufacturing of a long-term stable emulsion, one which does not sacrifice one property for another, can be frustrating.

One particularly good emulsifying system is described in U.S. Pat. No. 6,117,915 to Pereira et al., which issued on Sep. 12, 2000, and is assigned as its face to Croda, Inc. of Piscataway, N.J. This patent describes fatty alcohol phosphate ester emulsifier compositions which have been useful in numerous products. One material which is commercially available falling within the scope of this patent is CRODAFOS CES, which is a mixture of cetearyl alcohol, dicetyl phosphate and ceteth-10 phosphate in a fatty alcohol, which accounts for about 75% by weight, available from Croda, Inc., 300 Columbus Circle, Edison, N.J. 08837.

Despite being an excellent emulsifier, it has been found that when formulated in certain high oil content products such as, for example, hair relaxers, a phase inversion can occur during processing. A phase inversion is often characterized by a drop in viscosity. It was found that the products made using CRODAFOS CES generally exhibited excellent properties and in particular, excellent properties in terms of oil deposition despite inversion. However, manufacturers often do not like products that exhibit a phase inversion, particularly when not expected, and some are concerned that such phase inversions can yield undesirable properties as well. Certainly, phase inversions in some systems have that potential. However, substitution of a different emulsifier, one which might address the phase inversion issue, could also sacrifice the advantages in terms of the various properties. While the material may be more easily manufactured, the desirability of the end product is compromised.

Another material available from Croda, Inc. is sold under the trade name CRODA BLEND FHC. This material is a combination of CRODAFOS CES, PROCETYL AWS (PPG-5-7 ceteth-20), CRODACOL C-95 (cetyl alcohol NF) and a quat, in particular INCROQUAT BEHENYL TMS (benentrimonium methosulfate and cetearyl alcohol). HP-185 has been described for use in hair color products and in particular, a hair color cream known as HP-185. HP-185 contained approximately 10.75% fatty alcohol (4% CRODACOL C-95 and approximately 75% by weight of the CRODAFOS CES which is “inactive” nonphosphate containing fatty alcohol materials), which made up its oil phase.

However, the hair color material identified as HP-185 did not suffer from the phase inversion during manufacturing and does not contain a generally high oil content, i.e., greater than about 15% by weight of the formulation.

### SUMMARY OF THE INVENTION

In accordance with one aspect of the present invention, there is provided an emulsifying system which is useful in retarding phase inversion in systems which are susceptible to same.

In another aspect of the present invention there is provided an emulsifying system useful in high oil phase content products, products containing greater than about 15% of an oil phase, more preferably about 20% or more of an oil phase and even more preferably about 25% or more of an oil phase. Most preferably, a high oil phase content product in accordance with the invention will have about 30% or more of an oil phase. The personal care products produced using these emulsifying systems and methods of producing high oil content products with good oil deposition properties, and without an inversion during manufacturing are also contemplated.

A particularly preferred aspect of the present invention is a finished, oil-in-water emulsion produced without a phase inversion. In another particularly preferred embodiment of the present invention there is provided a finished emulsion, which is an oil-in-water emulsion in which the total oil content (excluding the primary emulsifier, the secondary emulsifier and any portion of the water phase) is at least 15% by weight of the finished emulsion.

Another preferred embodiment in accordance with the present invention is a finished, oil-in-water emulsion, which was made without a phase inversion, which has an oil phase content of about 15% or more by weight, which has desirable oil deposition properties.

In a particularly preferred embodiment, the present invention provides an oil-in-water emulsion based hair relaxer, depilatory or permanent wave product of the lye or no-lye type which is produced without a phase inversion, is a high oil content product or both, made with a primary and secondary emulsifier in accordance with the present invention. In a particularly preferred embodiment, the resulting emulsion based products exhibit advantageous oil deposition properties, often similar to those which result from a system which contains only the primary emulsifier.

### BRIEF DESCRIPTION OF THE DRAWINGS

FIG. 1 is a scanning electron microscope image of a hair sample relaxed using the formula described in Example 1.

FIG. 2 is a scanning electron microscope image of a hair sample relaxed with the formula of Example 2.

FIG. 3 is a scanning electron microscope image of a hair sample relaxed with the formula of Example 3.

FIG. 4 provides average dynamic advancing contact angle measurements for hair relaxers produced in accordance with Examples 1, 2, and 3.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

In accordance with one aspect of the present invention there is provided an emulsion system for producing oil-in-water emulsions which is comprised of an alkoxylated fatty acid, alkoxylated fatty amide, alkoxylated fatty alcohol or mixture thereof and a mixed fatty alcohol phosphate esters. In accordance with another aspect of the present invention there is provided an emulsion system for producing oil-in-water emulsions which consists essentially of an alkoxylated fatty acid, alkoxylated fatty amide, alkoxylated fatty alcohol, or mixture thereof and mixed fatty alcohol phosphate esters or simply “mixed phosphate esters.” For purposes of this invention, the mixed phosphate esters will be referred to as the “primary” emulsifier and the alkoxylated fatty acids, alkoxylated fatty amides, or alkoxylated fatty alcohols will be referred to as the “secondary” emulsifier.

The primary emulsifier is a fatty alcohol phosphate ester composition which is a mixture of both alkoxylated and nonalkoxylated phosphates of alkoxylated fatty alcohols and in particular, blends of mono- and di-esters phosphates of alkoxylated fatty alcohols. Preferred are those described in U.S. Pat. No. 6,117,915 to Pereira et al., which issued on Sep. 12, 2000, and is assigned as its face to Croda, Inc. of Edison, N.J., the text of which is hereby incorporated by reference.

In a particularly preferred embodiment, the primary emulsifier is comprised of between about 10% and about 70% by weight of a blend of mono- and di-ester phosphates of alkoxylated fatty alcohols containing between about 12 and 22 carbon atoms and alkoxylated with between about 1 and about 50 moles of an alkylene oxide consisting of ethylene oxide, wherein the mono- and di-ester ratio is between about 10:90 and about 90:10; and between about 90% and about 30% by weight of a blend of mono- and di-ester phosphates of non-alkoxylated fatty alcohols containing between about 12 and 22 carbon atoms, wherein the mono- and di-ester ratio is between about 10:90 and about 90:10. Particularly preferred material which can be used as the primary emulsifier in accordance with the present invention is CRODAFOS CES, which is a mixture of dicetyl phosphate and ceteth-10 phosphate in a fatty alcohol carrier, available from Croda, Inc., 300 Columbus Circle, Edison, N.J. 08837. The amount of primary emulsifier in accordance with the present invention depends on whether or not the product in question is a stand-alone emulsifier system or a finished formulation. However, in a finished formulation, the amount of primary emulsifier will generally range from between about 0.5% to about 10%, more preferably between about 1.0% to 7%, and most preferably about 2.0 to about 5%.

The secondary emulsifier comprises alkoxylated fatty acids, alkoxylated fatty alcohols, alkoxylated fatty amides, and mixtures thereof which include both ethoxy and propoxy units. More preferably, the number of ethoxy units will exceed the number of propoxy units. When considered in terms of the number of moles of each per mole of fatty acid or fatty alcohol, the ratio of ethoxy (“EO”) to propoxy (“PO”) unit should be a minimum of at least about 2:1, more preferably from about 2:1 to about 10:1. More preferably the range of ethyoxy to propoxy ranges from between about 2:1 to about 7:1 and most preferably from about 3:1 to about 6:1. Again, this is in terms of moles of ethoxy units to moles of propoxy units for each mole of fatty alcohol, fatty amide, or fatty acid.

The chain length for the fatty alcohol, fatty amide, or fatty acid portion of the secondary emulsifier can vary widely with the number of factors. However, preferably, a large portion of the emulsifier is made up of C₁₂-C₃₆ chain length fatty species, more preferably at least 40% of the fatty species will have a chain length of between C₁₂-C₃₆ and even more preferably at least about 50% will fall within this range. The fatty species can be substituted or unsubstituted and in particular, substituted with hydroxy groups such as, for example, 12 hydroxy stearic acid. The fatty components may also be saturated or unsaturated. Where unsaturated, preferably, no more than two unsaturated groups are found on any one molecule. The fatty groups may be branched or linear. The amount of secondary emulsifier will depend again on whether or not the formulation in question is a final product or a stand-alone emulsifying system. However, based on a formulation, preferably the amount of secondary emulsifier will range from between about 0.5% to about 10%, more preferably from between about 0.5% to about 5% and most preferably from between about 1% and about 3% by weight.

In a preferred embodiment, whether as a stand-alone emulsifying system as part of an oil-in-water emulsion or in a final product, the invention also includes at least one structuring agent. Structuring agents are generally oil phase materials (as used herein) used for viscosity modification and can include nonalkoxylated fatty alcohols, nonalkoxylated fatty acids, alkoxylated fatty acids or fatty alcohols which are homogeneous (all EO or all PO), waxes, hydrogenated glycerides and partially hydrogenated glycerides and the like. These can be found in final products in an amount of between about 0.5% to about 10%, more preferably between about 0.5% to about 5%, and most preferably between about 1% and about 3% by weight of the total formulation.

The use of an emulsifying system in accordance with the present invention including both a primary and secondary emulsifier as described herein, whether introduced together or introduced separately into a product, can retard phase inversions in susceptible systems. A phase inversion in accordance with the present invention is often characterized by a sudden decrease in viscosity, predominantly while the emulsion is going through the cool down stage. With emulsions containing high levels of oil phase, the phase inversion can also be characterized by an increase in viscosity accompanied by the appearance that the emulsion is coming apart. As stirring is continued however, the emulsion will reconstitute itself and yield a stable product. This is but a single way to measure an inversion. Any other which gives an unambiguous answer may also be used.

By the use of a primary and secondary emulsifier as described herein in an oil-in-water emulsion product, such inversions can be avoided.

Another aspect of the present invention is the use of this emulsifying system in high oil content oil-in-water emulsion products such as, for example, hair relaxers. A high oil content product is something with an oil phase by weight of, or in excess of 15% of the entire formulation, more preferably 20%, and even more preferably 25%, and most preferably about 30% or more by weight of the formulation. The oil phase in accordance with the present invention is made up of the components which are not the emulsifying system and are not water soluble or water dispersible at room temperature (when measured by standard stability testing). Typical components found in an oil phase include nonalkoxylated fatty acids or nonalkoxylated alcohols, ethoxylated fatty alcohols or acids, propoxylated fatty alcohols or acids, mineral oil, petrolatum, lanolin oil, waxes and the like. The structuring agents previously described are also part of the oil phase. The balance of the product is generally the water phase.

It has been found in the past to be difficult to provide such high oil content products, particularly those involved in hair relaxers, permanent waving products and depilatories (e.g., formulations often with harsh and/or corrosive chemicals) which are stable and provide the appropriate feel, usability, producibility and advantageous oil deposition properties. Products such as CRODAFOS CES have been found to provide advantageous oil deposition properties as measured by the techniques described in the aforementioned '915 patent. However, formulations with this as a primary emulsifier only can suffer from phase inversions. In certain systems, such phase inversions may provide advantageous properties. In others, however, such phase inversions could provide undesirable qualities. Moreover, unintended phase inversions often affect the desirability of a product in terms of processability.

In one embodiment, the fatty alcohol phosphate ester mixtures of the primary emulsifier of the invention include between about 10% and about 70% by weight of a blend of mono- and di-ester phosphates of alkoxylated fatty alcohols containing between about 12 and 22 carbon atoms and alkoxylated with between about 1 and about 50 moles of an alkylene oxide consisting of ethylene oxide, wherein the mono- and di-ester ratio is between about 10:90 and about 90:10; and between about 90% and about 30% by weight of a blend of mono- and di-ester phosphates of nonalkoxylated fatty alcohols containing between about 12 and 22 carbon atoms, wherein the mono- and di-ester ratio is between about 10:90 and about 90:10.

The fatty alcohol phosphate ester mixtures useful in accordance with the present invention are a blend of mono- and di-ester phosphates of alkoxylated and non-alkoxylated fatty alcohols containing between 12 and 22 carbon atoms. Preferred fatty alcohols contain between 14 and 20 carbon atoms. Most preferably, a fatty alcohol blend known as cetearyl alcohol is employed, which is a blend of cetyl and stearyl alcohols, which contain 16 and 18 carbon atoms, respectively.

The phosphate esters of the alkoxylated and non-alkoxylated fatty alcohols of the present invention may be formed by reacting alkoxylated and non-alkoxylated fatty alcohols, respectively, with phosphorous pentoxide (P₂O₅). The alkoxylated fatty alcohols preferably have between about 2 and about 20 moles of the alkoxylating moieties present for each fatty alcohol moiety and are preferably either polyethoxylated, polypropoxylated or both polyethoxylated and polypropoxylated. Therefore, preferred alkoxylated fatty alcohols for use in accordance with the present invention have the structural formula of Formula I: 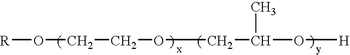
wherein R is a saturated or unsaturated, substituted or unsubstituted fatty moiety containing from 12 to 22 carbon atoms. X and Y are independently zero or integers from 1 to 50, inclusive, and the sum of X and Y is between 1 and 50, inclusive.

The non-alkoxylated fatty alcohols suitable for use in accordance with the present invention have the structural formula of Formula II:
R—OH (II)
R is the same as described above with respect to Formula I. As is well understood by those of ordinary skill in the art, fatty alcohols are derived from fatty acids, and for this reason, groups such as R are defined as fatty moieties. Fatty alcohols are often commercially prepared from a mixture of fatty acids and contain a mixture of fatty moieties. Therefore, in accordance with the present invention, R may represent a blend of fatty moieties.

Saturated, unsubstituted fatty moieties containing from 14 to 20 carbon atoms are preferred, and, as noted above, a 16 and 18 carbon atom fatty moiety blend, known as a cetearyl blend, is most preferred.

The alkoxylated fatty alcohol depicted in Formula I is prepared by the alkoxylation of the fatty alcohol of Formula II. In the above-depicted alkoxylated fatty alcohol of Formula I, X and Y are preferably independently selected from integers from 2 to 20, inclusive, with the sum of X and Y preferably being between 2 and 20, inclusive.

The alkoxylated fatty alcohols of Formula I are prepared by initially reacting, either sequentially, or in their mixed forms, the fatty alcohols of Formula II with an epoxide, preferably ethylene oxide, propylene oxide, or mixtures thereof, in the presence of an acidic or basic catalyst. It is typical of propylene oxide to branch upon opening of the epoxide ring. Catalysts suitable for this reaction are well-known in the art and include, for example, organic and inorganic alkalis such as alkali metal oxides and hydroxides, e.g., potassium hydroxide, sodium methoxide, sodium borohydride, protic and Lewis acids, e.g., boron trifluoride, stannic chloride and sulfuric acid. Amines, quaternary ammonium compounds, water and other acids may also be employed. Mixtures of catalysts may also be employed. Certain reactive substrates known in the art, for example, acetylenic alkanols, may eliminate the need for such catalysts.

Preferably, a basic catalyst is used in this reaction and most preferably from about 0.1 to about 2.0 weight % of potassium or sodium hydroxide, sodium methoxide, sodium borohydride or mixtures thereof, based on the weight of the fatty alcohol. The reaction is carried out under anhydrous conditions to avoid formation of by-products, and at a temperature which is preferably in the range of from about 110° C. to about 200° C., although higher temperatures may be utilized.

The reaction can be carried out at substantially atmospheric pressure, although it is preferably carried out in an autoclave at pressures of from about 10 psig to about 80 psig. The amount of ethylene oxide or propylene oxide introduced to the reaction zone, and the duration of reaction time, determines the numbers of moles of such components added to the fatty alcohol of Formula II, as is well known by those of ordinary skill in the art. In Formula I, X represents the number of moles of ethylene oxide which are incorporated into each alkoxylated fatty alcohol chain. Likewise, Y represents the number of moles of propylene oxide that are incorporated into the alkoxylated fatty alcohol chain. As will be readily appreciated by those of ordinary skill in the art, stoichiometric quantities of fatty alcohols, ethylene oxide and propylene oxide are reacted together, and stoichiometric quantities of the alkoxylated fatty alcohol and P₂O₅ are then reacted together to form the mono- and di-phosphate ester alkoxylated fatty alcohol blend.

For alkoxylation reactions in which the fatty alcohol is both ethoxylated and propoxylated, that is, when neither X nor Y is zero, the alkoxylation reaction is preferably carried out sequentially in that the fatty alcohol is first reacted with the propylene oxide and after complete reaction, the ethylene oxide is introduced into the reaction. After complete reaction of the ethylene oxide, an acid, e.g., phosphoric acid or acetic acid, is introduced into the reaction mixture to neutralize the basic catalyst.

The phosphate ester mixtures of the present invention, in addition to being a blend of alkoxylated and non-alkoxylated fatty alcohol phosphate esters, are also mono- and diester phosphate blends of both the alkoxylated and non-alkoxylated fatty alcohol phosphate esters. Thus, the alkoxylated fatty alcohol of Formula I, prepared as described above, is next reacted in a conventional phosphating reaction with P₂O₅ to form a mono- and diester phosphate alkoxylated fatty alcohol blend.

The phosphating reaction is typically performed by combining stoichiometric quantities of the alkoxylated fatty alcohol and the P₂O₅. As is well understood by those of ordinary skill in the art, the ratio of the two reagents will depend upon the ratio of mono- and diester phosphates desired. To obtain significant quantities of diester in the first place, a stoichiometric excess of P₂O₅ should be employed, with greater excess levels of P₂O₅ employed to increase the level of diester obtained. A 1:3 molar ratio of P₂O₅ to alkoxylated fatty alcohol is preferred.

The alkoxylated fatty alcohol is heated to a temperature between about 35° C. and about 90° C., and preferably at a temperature between about 50° C. and about 80° C., and then combined with mixing with P₂O₅ to form a reaction mixture. The alkoxylated fatty alcohol is a liquid at this temperature, therefore, a reaction solvent is not needed. The reaction is then allowed to continue until essentially complete, typically until about 10% or less of unreacted alkoxylated fatty alcohol and trace amounts of unreacted P₂O₅, now in the form of phosphoric acid, remain, usually about four hours. The reaction mixture is then recovered as a mono- and diester phosphate blend of alkoxylated fatty alcohols.

The alkoxylated fatty alcohol phosphate ester mixtures are then combined with a mono- and diester phosphate blend of non-alkoxylated fatty alcohols. The phosphate ester blend of non-alkoxylated fatty alcohols is prepared essentially the same as the phosphate ester blend of the alkoxylated fatty alcohols, by reacting stoichiometric quantities of the fatty alcohol of Formula II and P₂O₅ essentially in the same manner as described above for the alkoxylated fatty alcohol phosphate ester blend.

As noted above, mixed forms of fatty alcohols containing from 12 to 22 carbon atoms can be employed. Therefore, the resulting phosphate ester blends of alkoxylated and non-alkoxylated fatty alcohols can contain mixtures of alkoxylated and non-alkoxylated fatty alcohol phosphate esters containing from 12 to 22 carbon atoms.

The phosphate ester compositions of the present invention are then prepared by blending the mono- and di-phosphate ester blends of alkoxylated fatty alcohols with the mono- and diester phosphate blends of non-alkoxylated fatty alcohols. Quantities of the alkoxylated and non-alkoxylated phosphate esters are added to a stirred vessel and heated with mixing at a temperature between about 60° C. and about 90° C., and preferably at a temperature between 75° C. and 85° C., until a uniform homogeneous mixture is obtained, typically about 30 minutes.

The amount of alkoxylated fatty alcohol phosphate esters blended with non-alkoxylated fatty alcohol phosphate esters will depend upon the ultimate ratio of phosphate esters of alkoxylated and non-alkoxylated fatty alcohols desired. The emulsifier compositions of the present invention contain between about 10% and about 90% of alkoxylated fatty alcohol phosphate esters and between about 90% and about 10% of non-alkoxylated fatty alcohol phosphate esters. Preferred emulsifier compositions contain the ratio of alkoxylated fatty alcohol phosphate esters to non-alkoxylated fatty alcohol phosphate esters between about 20:80 and about 80:20, and more preferably between about 30:70 and about 70:30. The desired ratio is obtained by combining the alkoxylated fatty alcohol phosphate esters and non-alkoxylated fatty alcohol phosphate esters on a weight ratio basis.

The fatty alcohol phosphate ester mixtures of the present invention may be formulated as emulsifying waxes. Emulsifying waxes are essentially a blend of the emulsifier compositions of the present invention with a fatty alcohol containing from 12 to 22 carbon atoms.

Preferred emulsifying waxes in accordance with the present invention will be based upon one or more fatty alcohols containing from 14 to 22 carbon atoms. The cetearyl alcohol blend of 16 and 18 carbon atom fatty alcohols is most preferred.

Oil-in-water emulsions in accordance with the present invention are generally made by combining an oil phase, a water phase and an amount of an emulsifier system of primary and secondary emulsifiers effective to form an emulsion of the oil and water phase. Likewise, oil-in-water microemulsions in accordance with the present invention combine an oil phase, a water phase and an amount of an emulsifier effective to form a microemulsion of the oil and water phases. The fatty alcohol phosphate ester mixtures may be used as the emulsifier. However, any other emulsifier may be used to build the emulsion.

Typical emulsions contain an oil phase at a level between about 15 and about 80% by weight, preferably between about 17 and about 60% by weight, and more preferably between about 20 and about 40% by weight; and a water phase at a level between about 10% and about 90% by weight, preferably between about 20 and about 80% by weight, and more preferably between about 40% and about 70% by weight, based on the total emulsion weight.

For microemulsions, significantly higher levels of emulsifier are often used, so that the oil droplets formed are so small that the emulsion is transparent. Typically, the emulsifier is present at a level greater than or equal to that of the oil phase up to a level of about 300% by weight of the oil phase. A level of between about 150 and about 275% by weight of the oil phase is preferred, with a level of between about 225% and about 250% of the oil phase being more preferred. Such microemulsions typically contain an oil phase at a level of between about 5% and about 80% by weight, and preferably between about 20% and about 40% by weight. The water phase is typically at a level between about 20% and about 95% by weight, preferably between about 30 and about 70% by weight, and most preferably between about 40% and about 60% based on the total weight of the microemulsion.

The oil-in-water emulsions of the present invention are formulated utilizing techniques that are well-known in the art. Typically, all water-soluble ingredients are mixed together to form the water phase and all water-insoluble ingredients are mixed together to form the oil phase. The two phases are then combined with the emulsifier composition of the present invention and mixed until an emulsion is formed.

The microemulsion compositions of the present invention are formulated in a similar manner, particularly as described in co-pending and commonly owned U.S. patent application Ser. No. 08/052,557, filed Apr. 23, 1993, now abandoned, the disclosure of which is incorporated herein by reference. The emulsifier compositions of the present invention are substituted for the surface active agents described in that application.

The emulsion and microemulsion based topical preparations (collectively referred to as emulsions unless the context suggests otherwise) of the invention are formulated utilizing techniques that are well-known in the art. Typically, the water-soluble ingredients are dissolved in the water-phase and the water-insoluble ingredients are combined with the oil phase prior to formation of the emulsion. Typically, the ingredients are combined with mixing and the addition of heat if necessary until uniform, homogeneous phases are formed. The two phases are then combined with the addition of the emulsifier composition of the present invention to form an emulsion or microemulsion based topical preparation.

Those of ordinary skill in the art can readily identify whether a particular active agent is water-soluble or water-insoluble and therefore whether it should be included in the water phase or oil phase of the emulsion. Likewise, whether the topical preparation will be based on an emulsion, microemulsion, ointment or other delivery format is more or less an aesthetic determination based upon whether a milky, opaque product is desired, or whether a clear gel-like microemulsion is preferred. In selecting the microemulsion product form, potential skin irritation from the use of elevated levels of emulsifier and/or surfactant should be considered.

The topical preparations of the present invention, in addition to including one or more active ingredients in an oil-in-water emulsion or microemulsion may also include coloring agents, fragrances, proteins, salts, preservatives, essential oils, antiacne agents, thickeners, viscosity modifiers, sun-screen agents, and the like. These additional components may be added in various amounts as is well-known in the cosmetic formulation art. Such ingredients need not be added prior to the emulsion formation, but may instead be combined with the emulsion with mixing and the addition of heat if necessary until a uniform, homogeneous product is formed. These other ingredients can be provided in amounts known in the industry and generally between about 0.01 and about 10% by weight per ingredient. Hair relaxers, depilatories and wave products will often include lye or no-lye materials used in such applications. Sodium Hydroxide, Potassium hydroxide, and Lithium Hydroxide are examples and they can be used in amounts ranging from 0.05 to 5% w/w on an active basis. The actual amount of lye used will vary depending on the desired function of the final formula. For example, a moisturizing lotion may have only 0.1% KOH to bring the pH to about 5.5-6 while a relaxer may require between 1.5-2.5% NaOH to be effective.

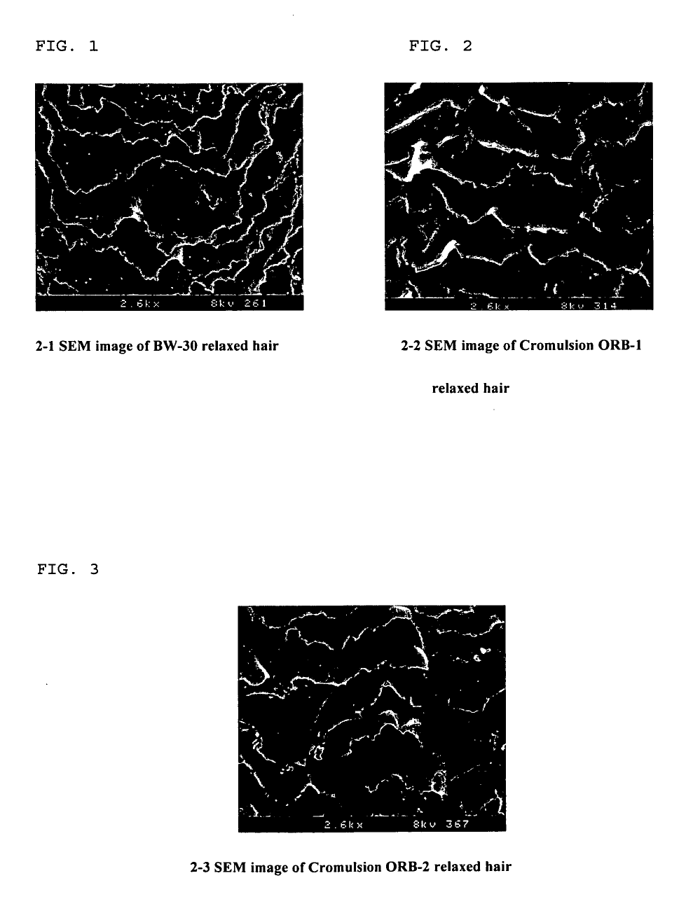

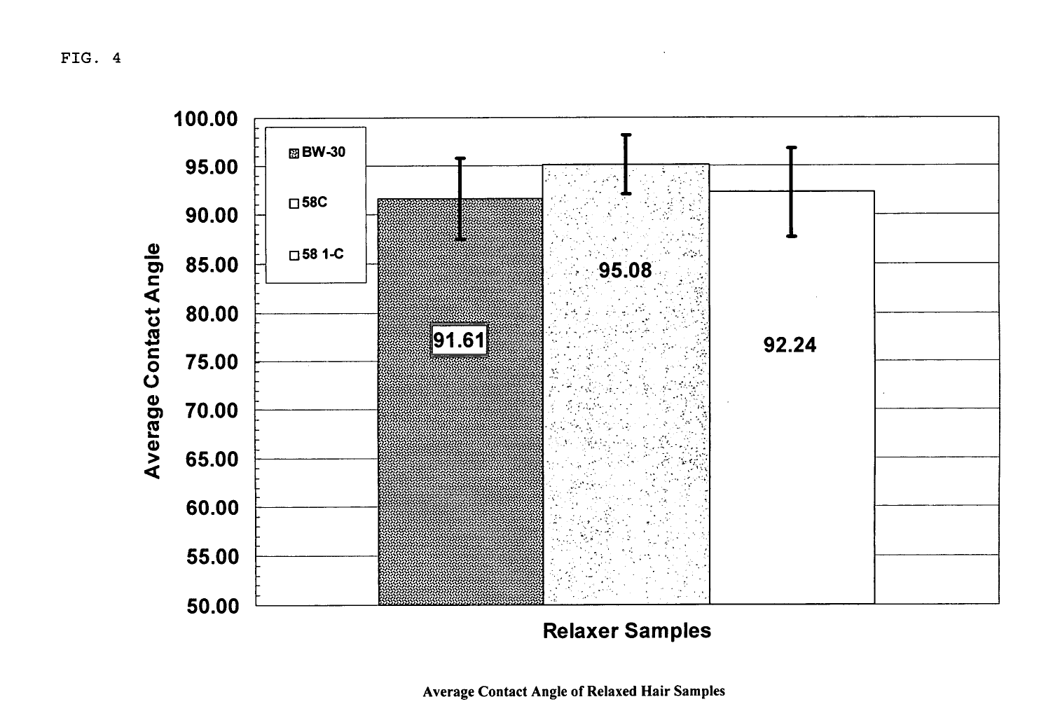

The hair relaxer product produced in accordance with Example 1, designated as BW-30 is successful in that it provides a good stable emulsion with advantageous oil deposition properties. However, during production, it exhibits a phase inversion. The BW-30 formulation includes approximately 7% CRODAFOS CES, a product of approximately 75% fatty alcohols and 25% mixed fatty alcohol phosphate esters as described herein. The oil phase included 21% petrolatum and 15% mineral oil. FIG. 4 shows the results of testing of the average dynamic advancing contact angle of hair fibers relaxed with BW-30 as prepared in accordance with Example 1. The average contact angle for BW-30 was 91.61 indicating that the hair surface is hydrophobic in nature. This suggests the significant deposition of oil from the relaxer system. Turning to FIG. 1, the scanning electron microscope image of hair relaxed with BW-30 shows the generally small extant of cuticle lift damage due to the relaxation process.

The formulation identified as ORB-1 relaxer was prepared in accordance with Example 2. This formula includes approximately 10% of Crodafos CES and 2% Procetyl AWS as illustrated in Example 2, which are primary and secondary emulsifiers, respectively, in accordance with the present invention. As shown in FIG. 4, the average dynamic advancing contact angle measurements ORB-1 relaxer, also referenced as “58C” was 95.08, even higher than that observed for BW-30. This indicates a slightly better contact angle than BW-30 and good relative oil deposition based on the hydrophic nature of the surface of the hair relaxed with that formulation. An SEM image of hair relaxed with ORB-1 is illustrated in FIG. 2 and it reveals a similar extent of cuticle lift damage due to relaxing. Thus, it was possible to obtain, through the use of the present invention, relatively comparable oil deposition and in some embodiments, without a phase inversion.

Finally, a third formulation is exemplified in Example 3. This formulation, like that exemplified in Example 2 included an emulsion system in accordance with the present invention. However, the degree of petrolatum was reduced from 21% to 15%. The average dynamic advance and contact angle measurements were similar to those observed for BW-30 and ORB-1 relaxer (92.24) (also referred to in FIG. 4 as 58-1C). The SEM image in FIG. 3 demonstrates relatively more severe cuticle damage. However, this may be due to the lower content of oil phase thus limiting the amount of oil which can be deposited.

Nonetheless, by the use of the present invention as illustrated with ORB-1, it is possible to obtain a hair relaxer system with high oil content and advantageous oil deposition properties and which, in some embodiments, is stable and provides comparable performance, without a phase-in version.

### EXAMPLE 1

| Ingredients | % |
|---|---|
| PART A | |
| CRODAFOS CES (Cetearyl Alcohol (and) Cetearyl | 7.0 |
| Phosphate) | |
| CRODACOL S-70 (Stearyl Alcohol) | 2.5 |
| Petrolatum | 21.0 |
| Mineral Oil (70ssu) | 15.0 |
| VOLPO S-2 (Steareth-2) | 0.5 |
| VOLPO S-10 (Steareth-10) | 2.5 |
| CRODACOL C-70 (Cetyl Alcohol) | 1.0 |
| Deionized Water | 39.2 |
| Propylene Glycol | 3.0 |
| Sodium Hydroxide | 2.1 |
| Deionized Water | 6.0 |
| pH = 13.5 ± 0.5 | |

Milled Viscosity=160,000 cps±10% (RVT Spindle TD@10 rpm@25° C.).

Procedure

Combine ingredients of Part A with mixing and heat to 75-80° C. Combine ingredients of Part B with mixing and heat to 75-80° C. Add Part B to Part A with mixing and cool to 40° C. Combine ingredients of Part C with mixing and add to Part A/B. Cool to desired milling temperature, mill product, and fill at desired fill temperature. During production, a phase inversion occurred. This resulting formulations are also referred to as “BW-30.”

### EXAMPLE 2

### (ORB-1 Relaxer)

INGREDIENTS

| | |
|---|---|
| PART A | |
| Crodafos CES(Cetearyl Alcohol & Ceteth-10 Phosphate & Dicetyl | 10.00 |
| Phosphate) | |
| Procetyl AWS (PPG-5 Ceteth-20) | 2.00 |
| Crodacol C-95 (Cetyl Alcohol) | 1.00 |
| Mineral Oil | 15.00 |
| Petrolatum | 21.00 |
| PART B | |
| Deionized Water | 40.90 |
| Propylene Glycol | 02.00 |
| PART C | |
| Deionized Water | 06.00 |
| Sodium Hydroxide Pellets 98% | 02.10 |
| Un-milled Viscosity: 168,900 cps | |
| (RVT Spindle #TD @ 10 RPM @ RT) | |

pH: 13.25

Procedure

Combine part A and heat to 65 C. Heat part B to 65 C. Add part B to part A. Allow emulsion to cool to 50 C. Add water bath. Premix part C and place in water bath to cool to room temperature. At 40 C. add ice cubes to emulsion water bath. Switch to side sweep blade. Slowly add part C. After complete addition, continue mixing for 30 minutes or until completely smooth and homogenous. No phase inversion occurred during production.

### EXAMPLE 3

INGREDIENTS

| | |
|---|---|
| PART A | |
| Crodafos CES(Cetearyl Alcohol & Ceteth-10 Phosphate & | 10.00 |
| Dicetyl Phosphate) | |
| Procetyl AWS (PPG-5 Ceteth-20) | 2.00 |
| Crodacol C-95 (Cetyl Alcohol) | 1.00 |
| Mineral Oil | 15.00 |
| Petrolatum | 15.00 |
| PART B | |
| Deionized Water | 46.90 |
| Propylene Glycol | 02.00 |
| PART C | |
| Deionized Water | 06. 00 |
| Sodium Hydroxide Pellets 98% | 02.10 |
| Un-milled Viscosity: 168,900 cps | |
| (RVT Spindle #TD @ 10 RPM @ RT) | |
| pH: 13.25 | |

Procedure

Combine part A and heat to 65 C. Heat part B to 65 C. Add part B to part A. Allow emulsion to cool to 50 C. Add water bath. Premix part C and place in water bath to cool to room temperature. At 40 C. add ice cubes to emulsion water bath. Switch to side sweep blade. Slowly add part C. After complete addition, continue mixing for 30 minutes or until completely smooth and homogenous. No phase inversion occurred during production.

### EXAMPLES 4-6

Three formulations were prepared which were identical to those described in Example 2 above with the following exception: Example 4—Procetyl AWS (PPG-5 Ceteth 20) was replaced with Incropol CS-20 (ceteareth-20) in an equivalent percentage of 2.05%; Example 5—Procetyl AWS was replaced with Procetyl 10 (RRG-10 Cetyl Ether) in an equivalent percentage of 2.056; Example 6—Procetyl AWS was replaced with about 1.05% of Incropol CS-20 and 1.05% Procetyl 10. They were prepared as described in Example 2. In each instance, an inversion was observed. A second round of each of experiments produced the same results. The use of Procetyl AWS, which is an ethoxylated and propoxylated fatty alcohol used as the secondary emulsifying agent in comparison produced no inversion.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A oil-in-water emulsion based cosmetic or personal care product comprising: a water phase, an oil phase in an amount which is at least about 15% by weight of the product, and which includes a primary emulsifier of mixed fatty alcohol phosphate esters and a secondary emulsifier which is an alkoxylated fatty alcohol, alkoxylated fatty amide or alkoxylated fatty acid which includes at least one ethoxy and at least one propoxy unit.

2. The cosmetic or personal care product of claim 1 wherein said emulsion is produced without a phase inversion.

3. The cosmetic or personal care product of claims 1 or 2 wherein said oil phase is present in an amount which is at least about 20% by weight of the product.

4. The cosmetic or personal care product of claim 3 wherein said oil phase is present in an amount which is at least about 25% by weight of the product.

5. The cosmetic or personal care product of claim 4 wherein said oil phase is present in an amount which is at least about 30% by weight of the product.

6. The cosmetic or personal care product of claims 1 or 2 wherein said secondary emulsifier includes more ethoxy units and than propoxy units.

7. The cosmetic or personal care product of claim 6 wherein said secondary emulsifier includes a ratio of ethoxy units to propoxy units of at least 2:1.

8. The cosmetic or personal care product of claim 7 wherein said secondary emulsifier includes a ratio of ethoxy units to propoxy units of between about 3:1 and about 6:1.

9. The cosmetic or personal care product of claims 1 or 2 wherein said primary emulsifier is provided in an amount of between about 0.5 and about 10% by weight of the product.

10. The cosmetic or personal care product of claim 9 wherein said primary emulsifier is provided in an amount of between about 1 and about 7% by weight of the product.

11. The cosmetic or personal care product of claims 1 or 2 wherein said secondary emulsifier is provided in an amount of between about 0.5 and about 10% by weight of the product.

12. The cosmetic or personal care product of claim 11 wherein said primary emulsifier is provided in an amount of between about 0.5 and about 5% by weight of the product.

13. A oil-in-water emulsion based cosmetic or personal care product comprising: a water phase, an oil phase and which includes a primary emulsifier of mixed fatty alcohol phosphate esters and a secondary emulsifier which is an alkoxylated fatty alcohol, alkoxylated fatty amide or alkoxylated fatty acid which includes at least one ethoxy and at least one propoxy unit wherein said emulsion is produced without a phase inversion.

14. The product of claim 13 wherein said emulsion would invert during production if produced without said secondary emulsifier.

15. The product of claim 13 wherein said oil phase is present in an amount which is at least about 25% by weight of the product.

16. The product of claim 15 wherein said oil phase is present in an amount which is at least about 30% by weight of the product.

17. The product of claim 16 wherein said secondary emulsifier includes more ethoxy units and than propoxy units.

18. The product of claim 17 wherein said secondary emulsifier includes a ratio of ethoxy units to propoxy units of at least 2:1.

19. The product of claims 13 or 17 wherein said primary emulsifier is provided in an amount of between about 0.5 and about 10% by weight of the product.

20. The cosmetic or personal care product of claim 19 wherein said primary emulsifier is provided in an amount of between about 1 and about 7% by weight of the product.

21. The cosmetic or personal care product of claims 13 or 17 wherein said secondary emulsifier is provided in an amount of between about 0.5 and about 10% by weight of the product.

22. The cosmetic or personal care product of claim 21 wherein said primary emulsifier is provided in an amount of between about 0.5 and about 10% by weight of the product.

23. A oil-in-water emulsion based cosmetic or personal care product comprising: a water phase, an oil phase in an amount which is at least about 25% by weight of the product, and which includes a primary emulsifier of mixed fatty alcohol phosphate esters in an amount of between about 0.5 and about 10% and a secondary emulsifier which is an alkoxylated fatty alcohol, alkoxylated fatty amide or alkoxylated fatty acid which includes at least one ethoxy and at least one propoxy unit and which includes more ethoxy units and than propoxy units in an amount of between about 0.5 and about 10%, and wherein said emulsion is produced without a phase inversion and wherein said product is a hair relaxer, depilatory or permanent waving product.

24. The product of claim 23 further comprising at least one structuring agent in an amount of between about 0.5 and about 10%.
